(19) United States
(12) Patent Application Publication
(10) Pub. No.: US 2008/0057568 A1
(43) Pub. Date: Mar. 6, 2008

(54) CULTURE APPARATUS

(76) Inventors: Kazutoshi KAN, Kawagoe (JP); Toyoshige Kobayashi, Hatoyama (JP)

Correspondence Address: MATTINGLY, STANGER, MALUR & BRUNDIDGE, P.C., 1800 DIAGONAL ROAD, SUITE 370, ALEXANDRIA, VA, 22314, US

(21) Appl. No.: 11/774,093

(22) Filed: Jul. 6, 2007

(30) Foreign Application Priority Data
Sep. 4, 2006 (JP) 2006-238924

(51) Int. Cl. C12M 1/38; C12M 1/36; C12M 3/00

(52) U.S. Cl. 435/286.5; 435/286.1; 435/303.1

(57) ABSTRACT

The invention provides a culture apparatus capable of setting preferable environments for each element of a small-sized culture chamber and manipulator, allowing an automatic culturing operation. The culture apparatus according to the invention is provided with a first chamber and a second chamber being a culture chamber. A device for producing moisture is arranged only within the second chamber. Accordingly, a difference between the humidity environment of the first chamber and that of the second chamber can be produced, and thus a manipulator arranged outside the second chamber, accessible to the second chamber can be prevented from being adversely affected by moisture.

### CLAIM OF PRIORITY

The present application claims priority from Japanese application JP 2006-238924 filed on Sep. 4, 2006, the content of which is hereby incorporated by reference into this application.

### BACKGROUND OF THE INVENTION

1. Field of the Invention

The present invention relates to a culture apparatus, and more particularly to a culture apparatus for tissue culturing (automatic culturing) of, for example, animal cells.

2. Background Art

In medical science- and medical care-related fields and the like, culturing of various types of cells separated from biologic bodies is being actively performed. In order to efficiently performing this type of cell culturing, the culture environment must be brought as close as possible to the environment within the biologic body in which this cell has resided before separation of the cell. Also, in the cell culturing, contamination must be prevented. Particularly, a human can be the largest contamination source, so the interposition of a human is desirably prevented as much as possible.

For example, Patent Document 1 has disclosed a culture apparatus provided with a first chamber and a second chamber for culturing cells. In this culture apparatus, the first chamber is equipped with a plurality of air lock type port openings; and a manipulator operating by remote control or automatic control is arranged in the first chamber. This manipulator is accessible to both the air lock type port openings and the second chamber. Accordingly, the contamination of the culture apparatus by a human can be eliminated.

[Patent Document] JP Patent Publication (Kokai) No. 2004-236514

However, in the culture apparatus disclosed in Patent Document 1, the through control of temperature and humidity environment has not been addressed. The humidity of environment for culturing cells within a culture apparatus is ordinarily 100%. Consequently, in the culture apparatus of Patent Document 1, mechanical devices having a movable part such as a motor, including the manipulator, are exposed to the environment of humidity of 100%, and thus dew condensation, resulting corrosions, and the like adversely affects the devices, which is not preferable.

To address these circumstances, the present invention has been devised, and provides a culture apparatus capable of setting preferable environments for each element of a small-sized culture chamber and manipulator, allowing an automatic culturing operation.

### SUMMARY OF THE INVENTION

To solve the above problem, a culture apparatus according to the present invention is provided with a first chamber and a second chamber being a culture chamber, and a device for producing moisture is arranged only within the second chamber. Accordingly, a difference can be produced between the humidity environment of the first chamber and that of the second chamber, and thus a manipulator arranged outside the second chamber, accessible to the second chamber can be prevented from being adversely affected by moisture.

The device for producing moisture maintains the humidity at a predetermined level using water supplied to a floor (bottom) within the culture chamber. Also, the device is provided with a heater for maintaining the temperature of this water at a predetermined level and a temperature sensor monitoring the water temperature; the heat production amount of the heater is controlled based on information of the temperature sensor. Further, when the water is supplied according to reduction thereof, it is also possible to deal with a culture processing over a relatively long period of time, and favorable culture environment can be maintained for a long period of time.

Also, a heater for heating culture vessels is arranged within the second chamber, allowing more stable maintenance of culture environment.

Further, regarding the manipulator, when a drive motor thereof is disposed at a section thereof other than a section (operating section) which enters the second chamber, the manipulator can be prevented from being affected by moisture.

More specifically, to solve the above problem, according to the present invention, there is provided a culture apparatus that cultures cells using culture vessels, the culture apparatus including: a first chamber; a device controlling a temperature within the first chamber; a second chamber housed in the first chamber, having a door section; a moisture producing device for humidifying the interior of the second chamber; a holding device housed in the second chamber for holding culture vessels housing cells; and a manipulator arranged outside the second chamber for putting the vessels through the door section in and out the second chamber. The humidity of the second chamber is higher than that of the first chamber.

This culture apparatus is further provided with a first heater housed in the second chamber, for heating the culture vessels.

This culture apparatus is further provided with a temperature maintaining device maintaining the interior of the first chamber at a predetermined temperature.

This culture apparatus is further provided with an air supplying device supplying air at constant temperature and at slightly higher pressure than atmosphere pressure, whereby the internal air leaks through a small gap in the first chamber.

The manipulator is provided with a drive motor in a section thereof other than a section which enters the second chamber.

One embodiment of the moisture producing device includes: a second heater for heating humidifying water supplied to the second chamber; a water temperature detecting device detecting a temperature of the humidifying water; and a heat amount controlling device controlling a heat amount of the second heater according to a water temperature detected by the water temperature detecting device, and may further include a water volume detecting device detecting a volume of the humidifying water; a humidifying water supplying device supplying humidifying water; and a water volume controlling device controlling supplying of water by the humidifying water supplying device based on a detection result of the water volume detecting device.

Another embodiment of the moisture producing device includes: a humidifying water circulating device, provided with a heater for heating humidifying water, and supplying and circulating humidifying water at a predetermined temperature to the second chamber; a water temperature detecting device detecting a temperature of the humidifying water; and a water temperature controlling device controlling based on a result of the water temperature detecting device, a heat production amount of the heater arranged in the humidifying water circulating device and thereby adjusting a temperature of the humidifying water.

The holding device is constituted of a shelf holding a plurality of culture vessels; the door section has a plurality of small doors separately openable and closable with respect to the shelf so that the plurality of culture vessels can be separately taken out.

The culture apparatus is further provided with a fan arranged within the second chamber, producing an air volume sufficient to stir air within the second chamber.

According to the present invention, there is provided a culture apparatus that cultures cells using culture vessels, the culture apparatus including: a first chamber; a second chamber housed in the first chamber, having a door section; a moisture producing device for humidifying the interior of the second chamber; a holding device housed in the second chamber for holding culture vessels housing cells; a temperature controlling device for heating the culture vessels to keep at constant temperature; and a manipulator arranged outside the second chamber for putting the vessel through the door section in and out the second chamber.

Also, according to the present invention, there is provided a culture apparatus that cultures cells using culture vessels, the culture apparatus including: a first chamber; a second chamber housed in the first chamber, having a door section; a door drive device for opening and closing the door section; a moisture producing device for humidifying the interior of the second chamber; a holding device housed in the second chamber, holding a culture vessel housing cells; and a manipulator arranged outside the second chamber for putting the vessels through the door section in and out the second chamber, wherein the door section is a sliding door, opening and closing thereof being controlled by the door drive device so as to cover an opening arranged in the second chamber; and the manipulator includes: an operating section acting on the culture vessel; a drive section driving the operating section; and a supplementary member for ensuring a distance between the operating section and the drive section.

Other features of the present invention will be apparent from the following description of preferred embodiments and the accompanying drawings.

According to the inventive culture apparatus, preferable environment can be set for each element of a small culture chamber and manipulator, allowing an automatic culturing operation.

### BRIEF DESCRIPTION OF THE DRAWINGS

FIG. 1 is a view illustrating a schematic configuration of a culture apparatus 1 according to a first embodiment;

FIG. 2 is a view illustrating a state where a door 3 of a culture chamber 2 according to the first embodiment is open;

FIG. 3 is a view illustrating a schematic configuration of a culture apparatus 1 according to a second embodiment;

FIG. 4 is a view illustrating a schematic configuration in which the culture apparatus 1 according to the second embodiment is provided with a device (automatic liquid level maintaining device) supplying humidifying water;

FIG. 5 is a view illustrating a configuration in which the culture apparatus 1 according to the second embodiment is provided with another humidity production device different from that of the first embodiment;

FIG. 6 is a view illustrating a schematic configuration of a culture apparatus 1 according to a third embodiment; and

FIG. 7 is a view illustrating a configuration in which the culture apparatus 1 according to the third embodiment is provided with another door section different from that of the first embodiment.

### DETAILED DESCRIPTION OF THE PREFERRED EMBODIMENTS

Specific descriptions of embodiments (automatic culture apparatus) of a culture apparatus according to the present invention will be given below with reference to the drawings. It should be noted that configurations illustrated in the drawings are merely exemplary of the present invention, and are not be construed to limit the scope of the present invention.

### First Embodiment

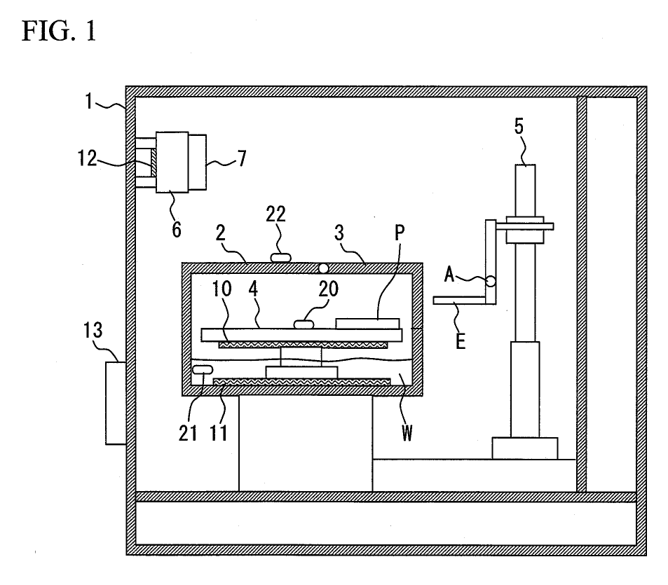

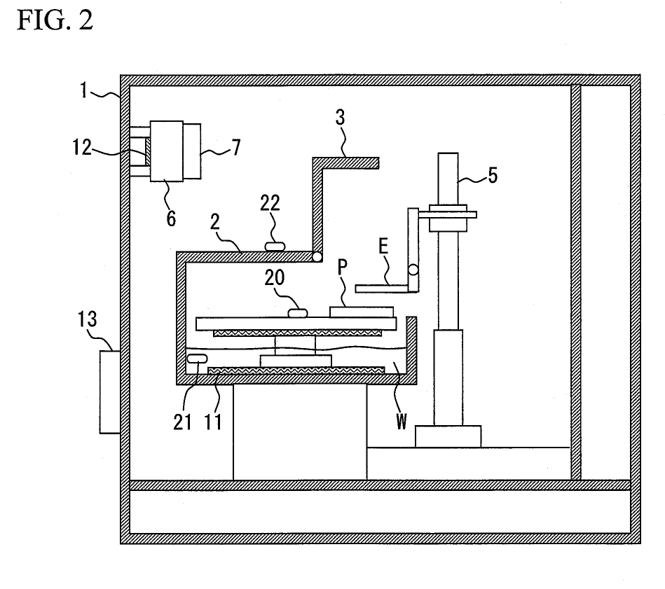

FIGS. 1 and 2 each illustrate a schematic configuration of an automatic culture apparatus according to a first embodiment of the present invention.

Referring to FIG. 1, within an automatic culture apparatus 1 being a first chamber, there are arranged: a culture chamber 2 being a second chamber constituting a closed space, having a door section 3; and a manipulator 5 for handling a small-sized culture vessel P and performing an operation such as replacement of culture medium. The outer wall of the automatic culture apparatus 1 is desirably composed of a heat-insulating material so that leaking of the internal heat is reduced to a minimum. Also, heat-insulating material, metal or the like can be used for the outer wall of the culture chamber 2. The use of a heat-insulating material for the outer wall has an advantage in that the internal heat of the culture chamber 2 hardly leaks and thus the internal temperature thereof can be controlled independently. The use of metal or the like for the outer wall has an advantage in that heat in the exterior of the culture chamber 2 can easily be transmitted to the interior thereof and at the same time, heat in the interior of the culture chamber 2 can also be transmitted to the exterior thereof and thus both the temperature environments of the first chamber and second chamber can easily be controlled at a culture temperature.

The first chamber is further provided with a heat radiator 6, a fan 7 and a heater 12 for maintaining the interior of the automatic culture apparatus 1 at a predetermined temperature. A temperature of the first chamber is measured by a temperature sensor 22 arranged in the outer side of the culture chamber 2; and the heat production amount of the heater 12 is controlled by an external controller (not illustrated) so that the temperature of the first chamber is maintained at a predetermined temperature: in this case, at a temperature during culturing (culture temperature: 37° C., for example).

The culture chamber 2 is provided in the interior thereof with a turn table 4 receiving the small-sized culture vessels P, a heater 10 for heating the turn table 4, and a temperature sensor 20 detecting a temperature of the turn table. The heat production amount of the heater 10 is controlled based on a temperature signal from the temperature sensor 20 by an external controller (not illustrated) so that the turn table 4 (and the small-sized culture vessel P) is maintained at a predetermined temperature: in this case, at the temperature during culturing.

The culture chamber 2 is provided in a bottom surface thereof with water W used for humidification, a heater 11 for heating the water W, and a temperature sensor 21 detecting a temperature of the water. The heat production amount of the heater 11 is controlled based on a temperature signal from the temperature sensor 21 by an external controller (not illustrated) so that the water W is maintained at a predetermined temperature: in this case, at the temperature during culturing.

The manipulator 5 is provided with a drive section (actuator A) for ensuring flexibility required for operation, and an end effector E.

In a state where culturing is under way with the small-sized culture vessel P placed within the culture chamber 2, the interior of the culture chamber 2 is maintained at the culture temperature and the water on the floor surface of the culture chamber 2 is being vaporized. Further, since the external atmosphere of the culture chamber 2 is maintained at a culture temperature by the heater 12 arranged within the culture apparatus 1, the interior of the culture chamber 2 is maintained at the culture temperature and at a humidity of 100%. Further, since the first chamber is isolated from the culture chamber 2 (second chamber) by the door section 3, the humidity of the culture chamber 2 is higher than that of the first chamber.

As illustrated in FIG. 1, the manipulator 5 is disposed outside the culture chamber 2, and the door 3 of the culture chamber 2 is being closed. Thus, the humidity of the interior of the culture chamber 2 does not affect the exterior thereof. Accordingly, the problems such as dew condensation on the manipulator 5 and resulting corrosions can be solved.

The culture apparatus 1 is also provided with an air supplying section 13 that supplies clean and constant-temperature air from the outside at a pressure slightly higher than atmosphere pressure; thus, the internal air leaks through a minor gap (not illustrated) of the culture apparatus 1. Consequently, a small amount of air leaks at all times and thus the air within the culture apparatus 1 is replaced bit by bit.

When an operation such as replacement of culture media takes place, as illustrated in FIG. 2, the door 3 of the culture chamber 2 is opened. This opening and closing operation of the door 3 is performed by a drive section (not illustrated) arranged outside the culture chamber 2 based on an instruction from an external controller (not illustrated). As illustrated in FIG. 2, with the door 3 opened, the manipulator 5 moves; and the end effector E holds the small-sized culture vessel P and a predetermined operation is performed in the interior or exterior of the culture chamber 2. When replacement of culture media is performed, culture media is supplied via a tube (not illustrated) extending to the end effector E, and the waste liquid is collected.

While the door 3 is open, the internal moisture leaks to the first chamber. However, since it does not take much time to perform the replacement of culture media, the humidity within the culture apparatus 1 will never change to the same level (100%) as the interior of the culture chamber 2. Further, the leaked moisture is gradually lessened by supplying clean air into the culture apparatus 1 to recover the original humidity. Accordingly, adverse effects of the leaked moisture can be neglected.

After the operation, the small-sized culture vessel P is returned to its original position, and the manipulator 5 retreats, and the door 3 is closed, and the culture environment is recovered.

According to the automatic culture apparatus 1 having the above described configuration and function, the 100% humidity environment preferable for the small-sized culture vessel P can be provided and at the same time the low humidity environment preferable for the manipulator 5 used for operation can also be maintained.

### Second Embodiment

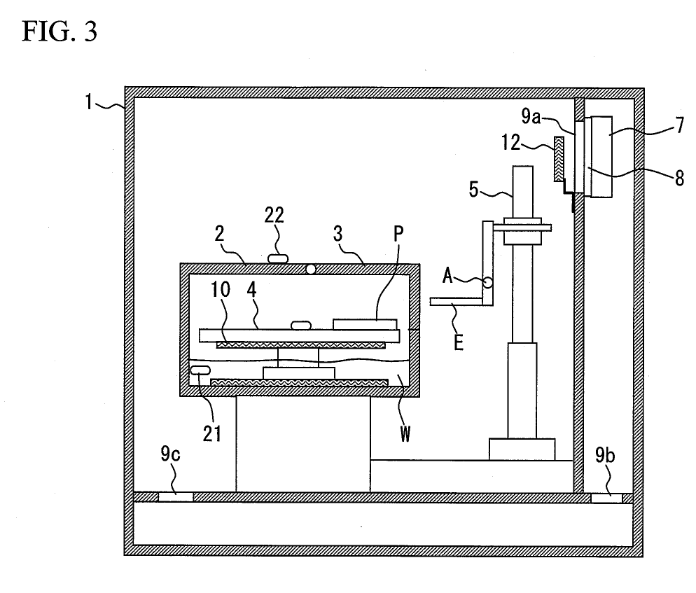

A second embodiment of the present invention will be described below with reference to FIGS. 3 to 5. FIG. 3 illustrates a schematic configuration of an automatic culture apparatus 1 according to the second embodiment of the present invention. In the automatic culture apparatus of FIG. 3, the configuration of a temperature maintaining device within the culture apparatus 1 is different from that of the first embodiment.

Referring to FIG. 3, gaps 9a, 9b and 9care arranged in walls of each chamber within the culture apparatus 1. In FIG. 3, a fan 7, filter 8 and heater 12 are arranged in the gap 9a; the fan 7 sends air to the heater 12. The heat production amount of the heater 12 is as with FIG. 1, controlled from the outside based on temperature information of the temperature sensor 22 arranged outside the culture chamber 2 so that a constant temperature is obtained. When this state is maintained, the air is circulated via the gaps 9b and 9cto reach gradually to a constant temperature; and at the same time, the internal microscopic particles are captured by the filter to allow raising of the cleanness within the culture apparatus 1.

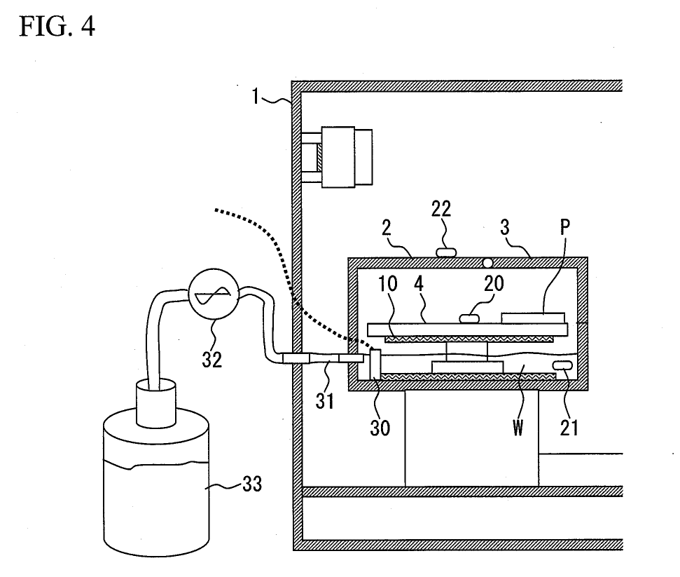

The automatic culture apparatus according to the second embodiment is as illustrated in FIG. 4, provided with a device automatically maintaining at a constant level the amount of water residing on the floor surface of the culture chamber 2 for humidity maintenance. More specifically, a water level detection sensor 30 monitors a level of water, and detects an amount of water which decreases when evaporation occurs and when humidity leaking at the time of opening the door 3 causes water to evaporate; and an external controller (not illustrated) determines a water supplying amount; and a pump 32 is driven to supply water W via a supplying tube 31 from an external water reserver tank 33. Typically, tissue culturing takes several weeks of culture period, so the capability of automatically supplying water over a long period is a function required for the automatic culture apparatus.

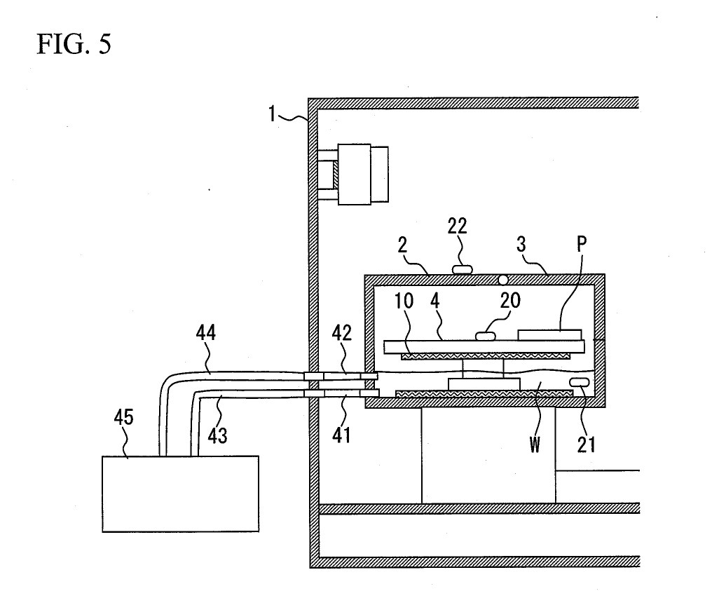

The automatic culture apparatus according to the second embodiment is as illustrated in FIG. 5, also provided with a humidity production device having a configuration different from that of the first embodiment. Referring to FIG. 5, heated water on the floor surface of the culture chamber 2 enters a constant temperature water unit 45 called a circulator via discharging water side tubes 41 and 43, and passes through a temperature adjustment system therein and a pump, and returns to the interior of the culture chamber 2 via supply side tubes 44 and 42. Accordingly, the water is circulated, and heated by a heater (not illustrated) arranged within the circulator 45 to maintain the water temperature at a constant level, and at the same time the humidity within the culture chamber 2 is maintained at 100% by causing the water to evaporate. Also, the water temperature within the culture chamber 2 is monitored by a temperature sensor 21 arranged within the culture chamber 2, and when the water temperature is lower than a predetermined level, the heat production amount of the heater of the circulator 45 is adjusted by an external controller (not illustrated), whereby the temperature can be efficiently controlled. In this case, the temperature sensor may be arranged within the circulator 45 to control the water temperature.

### Third Embodiment

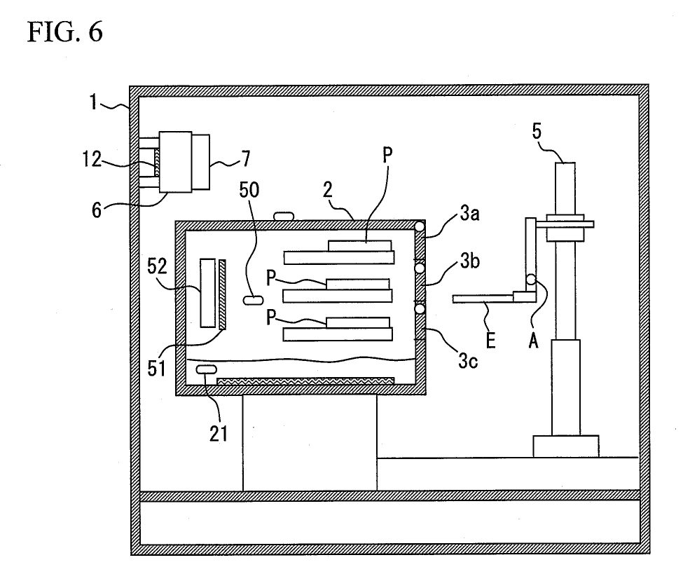

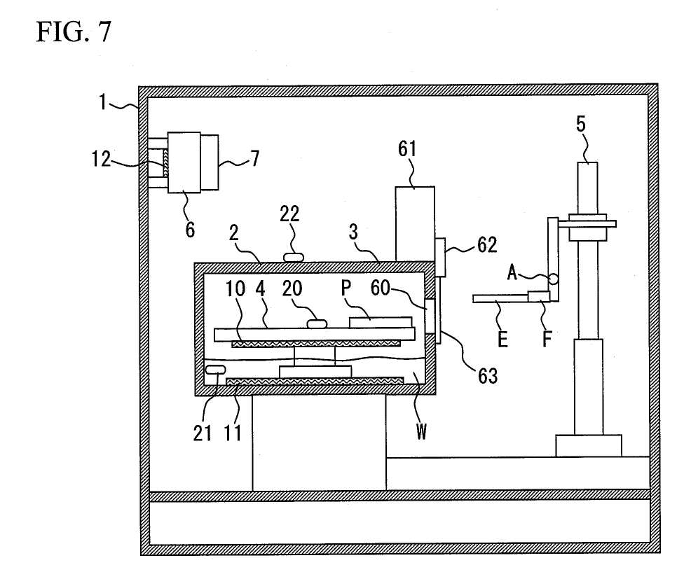

An automatic culture apparatus according to a third embodiment of the present invention will be described with reference to FIGS. 6 to 7. FIG. 6 illustrates a configuration of an automatic culture apparatus 1 different from that of the first embodiment in the method of receiving the small-sized culture vessel P. Referring to FIG. 6, the culture chamber 2 is provided with multiple shelves receiving a plurality of small-sized culture vessels P, and respective shelves are provided with doors 3a, 3b and 3c independently openable and closable. Instead of being separately opened, these doors may be opened all at once, or may be constituted of a single door. However, when the doors are separately opened and closed, the opening area of the culture chamber 2 can be reduced and thus adverse effects of humidity on the manipulator 5 can be comparatively reduced.

Further, in order to stir the internal air to reduce unevenness of the internal humidity and at the same time to maintain the temperature within the culture chamber 2 at a predetermined culture temperature, the culture chamber 2 is provided with a fan 52 producing an appropriate air volume, a heater 51 for maintaining the temperature at a predetermined culture temperature, and a temperature sensor 50 used for control. In this case, though not illustrated herein, in order to prevent humidity irregularity, there may be arranged a fin for dispersing and guiding the air sent by the fan 52, and a fixed rectifier board.

When a processing such as replacement of culture media is performed in the small-sized culture vessel P, for example, the door 3a is opened, and the end effector E of the manipulator 5 enters the interior of the culture chamber 2 and grasps the small-sized culture vessel P and then the processing is performed in the interior of the culture chamber 2 or after transferring the small-sized culture vessel P to the outside. After completion of the processing, the small-sized culture vessel P is placed in its original position, and the manipulator 5 retreats from the culture chamber 2. With another small-sized culture vessel P in another position, also, the corresponding door is similarly opened to perform the processing. Accordingly, the interior of the culture chamber 2 of a humidity of 100% is completely separated from the interior of the culture apparatus 1 where the manipulator 5 is placed, thus maintaining the environments adapting to the respective functions.

FIG. 7 illustrates an automatic culture apparatus 1 having a door of a structure different from that of the first embodiment. Referring to FIG. 7, the partition wall of the culture chamber 2 is provided with a gap (opening) 60, and there is a door 63 (for example, a sliding door) arranged so as to cover the gap. A movable section 62 of this door 63 is moved vertically by a vertically driving mechanism 61 to vertically move the door 63 secured in an integrated manner to the movable section 62, whereby a door function is provided. Corresponding to this door mechanism, the manipulator 5 is provided with a supporting member (supplementary member) F between an end effector E and an actuator A driving the effector E; when the door 6 is opened and the end effector E is made to enter the interior of the culture chamber 2, the actuator A which is the drive section can be placed at a longest distance from the interior of the culture chamber 2. In this way, there is used a structure in which the supporting member F allows the end effector E to be placed at a certain or more distance from the actuator A. Consequently, the actuator A of the manipulator that is particularly vulnerable to adverse effects of humidity and the like, can be placed at a longest distance from the high humidity environment, and thus the adverse effects of humidity can be prevented more efficiently. As evident from comparison with the structure of FIG. 3, the distance between the actuator and end effector can be lengthened in the structure of FIG. 7.

Though not illustrated, when a moisture absorbent material absorbing moisture leaking during opening of the door is arranged at a predetermined position within the culture apparatus 1, an environment more suitable for the manipulator 5 (particularly, actuator A) can also be maintained.

Also, when the actuator A is covered with a protective member such as resin, the adverse effects of humidity can be further prevented.

Further, in the first to third embodiments, water is supplied to the floor surface (bottom surface) of the culture chamber 2 and heated to obtain a humidity of 100%, but the present invention is not limited thereto; air of a humidity of 100% may be supplied to the culture chamber 2. In this case, there can be used a configuration in which the culture chamber 2 is provided with a device monitoring an internal humidity, and air of a culture temperature, containing moisture is sent to the interior of the culture chamber 2 based on a result thereof.

According to the above described culture apparatus of the present embodiments, the first chamber and the culture chamber (second chamber) are separated; the moisture producing device is arranged only in the second chamber; and both the chambers are maintained at a culture temperature. Accordingly, the small-sized culture vessel can be placed (maintained) in the environment of the culture temperature and a humidity of 100%, and the manipulator used for culturing operation can be placed in the environment of the culture temperature and a low humidity. Consequently, the entire manipulator can be prevented from being exposed to a high humidity area, thus allowing prevention of corrosions and the like of the manipulator. Also, since the drive section of the manipulator affected by humidity in the manipulator is arranged at a position other than the section thereof functioning in the high humidity area, e.g., the end effector, adverse effects of humidity can be further reduced.

Further, since the heater is also arranged in the turn table, the temperature control of the turn table (and the culture vessels) can be quickly performed, so the temperature can be maintained at a constant level even during opening/closing of the door. Also, water for supplying moisture can be automatically supplied and thus the environment can be automatically maintained over a culturing period.

Also, since the temperature maintaining device for maintaining the interior of the first chamber at a predetermined temperature is arranged, the first chamber and second chamber can be controlled at the same temperature.

Further, the culture apparatus according to the present embodiment is provided with the air supplying device supplying air at a constant temperature and at slightly higher pressure than atmosphere pressure and thus the internal air leaks through a small gap of the first chamber. Accordingly, even when the door section is open and the moisture of the second chamber leaks to the first chamber, air having no moisture is supplied to the first chamber, thus allowing suppression of adverse effects of moisture on the manipulator.

When the manipulator is provided with the drive motor in a section thereof other than a section entering the second chamber, a distance between the motor and culture vessel can be ensured, allowing suppression of adverse effects of moisture on the motor.

Concerning the regenerative medicine which cultures cells to utilize them for medical treatment or to examine them, there have been reported examples which exhibit innovative accomplishments at the laboratory level. However, in order for the regenerative medicine to popularize as a typical medical treatment, it is an important point how safely high-quality cultured tissues are efficiently produced. For this, not only culturing by human hands but also efficient automatic culturing is needed. In order to set it up in industry, the establishment of the automatic culturing technology is urgently needed. In the automatic culturing technology, a culturing processing by machine is indispensable; in this case, the environment required for culturing is not always identical to the placement environment required for the machine. Thus, the applicability of the present invention which can perform processings while maintaining environments suitable for each element is large.

What is claimed is:

1. A culture apparatus that cultures cells using a culture vessel, the culture apparatus comprising:
a first chamber;
a device controlling a temperature within the first chamber;
a second chamber housed in the first chamber, having a door section;
a moisture producing device for humidifying the interior of the second chamber;
a holding device housed in the second chamber for holding a culture vessel housing cells; and
a manipulator arranged outside the second chamber for putting the culture vessel through the door section in and out the second chamber.

2. The culture apparatus according to claim 1, further comprising a first heater housed in the second chamber, for heating the culture vessel.

3. The culture apparatus according to claim 1, further comprising a temperature maintaining device maintaining the interior of the first chamber at a predetermined temperature.

4. The culture apparatus according to claim 1, further comprising an air supplying device supplying air at a constant temperature and at a pressure higher than atmosphere pressure, whereby air within the first chamber leaks through a small gap in the first chamber.

5. The culture apparatus according to claim 1, wherein the manipulator is provided with a drive motor in a section thereof other than a section which enters the second chamber.

6. The culture apparatus according to claim 1,
wherein the moisture producing device comprises:
a second heater for heating humidifying water supplied to the second chamber;
a water temperature detecting device detecting a temperature of the humidifying water; and
a heat amount controlling device controlling a heat amount of the second heater according to a water temperature detected by the water temperature detecting device.

7. The culture apparatus according to claim 6,
wherein the moisture producing device further comprises:
a water volume detecting device detecting a volume of the humidifying water;
a humidifying water supplying device supplying humidifying water; and
a water volume controlling device controlling supplying of water by the humidifying water supplying device based on a detection result of the water volume detecting device.

8. The culture apparatus according to claim 1,
wherein the moisture producing device comprises:
a humidifying water circulating device, provided with a heater for heating humidifying water, and supplying and circulating humidifying water at a predetermined temperature to the second chamber;
a water temperature detecting device detecting a temperature of the humidifying water; and
a water temperature controlling device controlling based on a result of the water temperature detecting device, a heat production amount of the heater arranged in the humidifying water circulating device and thereby adjusting a temperature of the humidifying water.

9. The culture apparatus according to claim 1, wherein
the holding device is constituted of a shelf holding a plurality of culture vessels; and
the door section has a plurality of small doors separately openable and closable with respect to the shelf so that the plurality of culture vessels can be separately taken out.

10. The culture apparatus according to claim 1, further comprising a fan arranged within the second chamber, producing an air volume sufficient to stir air within the second chamber.

11. The culture apparatus according to claim 1, wherein the humidity of the second chamber is higher than that of the first chamber.

12. A culture apparatus that cultures cells using culture vessels, the culture apparatus comprising:
a first chamber;
a second chamber housed in the first chamber, having a door section;
a moisture producing device for humidifying the interior of the second chamber;
a holding device housed in the second chamber for holding culture vessels housing cells;
a temperature controlling device for heating the culture vessels to keep at constant temperature; and
a manipulator arranged outside the second chamber for putting the culture vessels through the door section in and out the second chamber.

13. A culture apparatus that cultures cells using a culture vessel, the culture apparatus comprising:
a first chamber;
a second chamber housed in the first chamber, having a door section;
a door drive device for opening and closing the door section;
a moisture producing device for humidifying the interior of the second chamber;
a holding device housed in the second chamber for holding a culture vessel housing cells; and
a manipulator arranged outside the second chamber for putting the culture vessel through the door section in and out the second chamber,
wherein the door section is a sliding door, opening and closing thereof being controlled by the door drive device so as to cover an opening arranged in the second chamber, and
wherein the manipulator includes: an operating section acting on the culture vessel; a drive section driving the operating section; and a supplementary member for ensuring a distance between the operating section and the drive section.
